(19) United States
(12) Patent Application Publication
(10) Pub. No.: US 2026/0365655 A1
(43) Pub. Date: Nov. 19, 2026

(54) NEW USE OF JANUS KINASE (JAK)-DEGRADING AGENT BASED ON PROTEOLYSIS TARGETING CHIMERA (PROTAC)

(71) Applicant: SHANGHAI SKIN DISEASE HOSPITAL, Shanghai (CN)

(72) Inventors: Zhongjian CHEN, Shanghai (CN); Junchao WU, Shanghai (CN); Zongguang TAI, Shanghai (CN); Quangang ZHU, Shanghai (CN); Lisha LI, Shanghai (CN)

(21) Appl. No.: 19/118,873

(22) PCT Filed: Jun. 5, 2023
(86) PCT No.: PCT/CN2023/098298

§ 371 Date: Apr. 7, 2025

(30) Foreign Application Priority Data
Nov. 24, 2022 (CN) 202211482126.8

(51) Int. Cl. C07D 487/04; A61K 31/519; A61P 17/00

(52) U.S. Cl. CPC C07D 487/04; A61K 31/519; A61P 17/00

(57) ABSTRACT

Provided is a new use of a compound shown in a formula (I), as a Janus kinase (JAK) inhibitor based on proteolysis targeting chimera (PROTAC), in a treatment of an inflammatory skin disease. A JAK inhibitor (JAKI) prepared from the compound, or a stereoisomer or optical isomer, a pharmaceutically acceptable salt, a prodrug, or a solvate thereof, or a pharmaceutical Composition including any of the above compounds, and an optional pharmaceutically acceptable carrier blocks the signaling of the JAK pathway through the targeted degradation of JAK, and thus has the potential to treat atopic dermatitis (AD) or related skin diseases involved by JAK. Therefore, the JAKI can improve an inflammatory skin disease by inhibiting JAK. This application has developed a new use for JAK-PROTAC compounds, especially a use in the treatment of diseases such as AD, psoriasis, vitiligo, and alopecia areata.

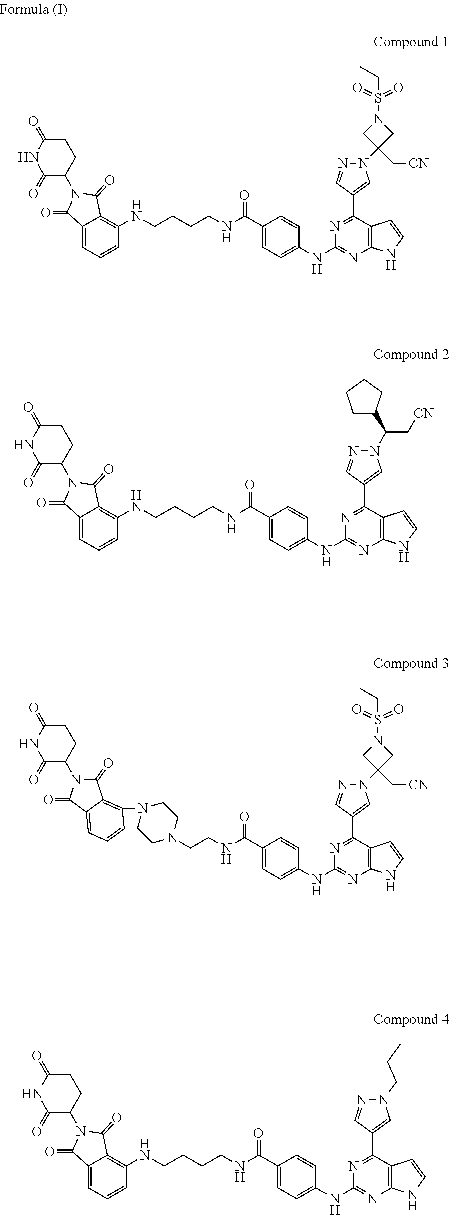 [SMILES: CCS(=O)(=O)N1CC(CC#N)(n2cc(-c3nc(Nc4ccc(C(=O)NCCCCNc5cccc6c5C(=O)N(C5CCC(=O)NC5=O)C6=O)cc4)nc4[nH]ccc34)cn2)C1 ; N#CC[C@H](C1CCCC1)n1cc(-c2nc(Nc3ccc(C(=O)NCCCCNc4cccc5c4C(=O)N(C4CCC(=O)NC4=O)C5=O)cc3)nc3[nH]ccc23)cn1 ; CCS(=O)(=O)N1CC(CC#N)(n2cc(-c3nc(Nc4ccc(C(=O)NCCN5CCN(c6cccc7c6C(=O)N(C6CCC(=O)NC6=O)C7=O)CC5)cc4)nc4[nH]ccc34)cn2)C1 ; CCCn1cc(-c2nc(Nc3ccc(C(=O)NCCCCNc4cccc5c4C(=O)N(C4CCC(=O)NC4=O)C5=O)cc3)nc3[nH]ccc23)cn1]

### CROSS REFERENCE TO RELATED APPLICATION

This patent application is a national stage application of International Patent Application No: PCT/CN2023/098298, filed on Jun. 5, 2023, which claims the benefit and priority of Chinese Patent Application No. CN202211482126.8 filed with the China National Intellectual Property Administration on Nov. 24, 2022, the disclosure of which is incorporated by reference herein in its entirety as part of the present application.

### TECHNICAL FIELD

The present disclosure belongs to the field of biomedicine, and specifically relates to a Janus kinase (JAK)-degrading agent based on proteolysis targeting chimera (PROTAC) and a new use thereof.

### BACKGROUND

Atopic dermatitis (AD), also known as eczema, is an inflammatory skin disease, with a global incidence of about 10%. AD mostly occurs in the early childhood, and sometimes occurs in the adolescence or adulthood. AD may generally develop into a chronic or recurrent disease. AD patients experience itchy skin, which is characterized by the impaired barrier function. The skin of AD patients is highly susceptible to secondary bacterial, viral, and fungal infections.

The existing studies on the inflammatory signaling in AD mainly focus on the Janus kinase (JAK)-signal transducer and activator of transcription (STAT) signaling pathway. The JAK-STAT signaling pathway is the core of cytokine receptor signaling. The JAK-STAT signaling pathway is composed mainly of the following three components: tyrosine kinase-associated receptor, tyrosine kinase (JAK), and transcription factor (STAT). JAK is the center of intracellular signaling for type-I and type-II cytokine receptors. The JAK family includes four tyrosine kinases, namely, Janus kinase 1 (JAK1), Janus kinase 2 (JAK2), Janus kinase 3 (JAK3), and tyrosine kinase 2 (Tyk2). STAT is short for signal transducer and activator of transcription. The JAK-STAT signaling pathway allows external signal stimuli to be transduced into cells, which plays an important role in both the cell signal transduction and the occurrence of various diseases such as immune-mediated diseases, cancer, and Parkinson's disease. It has been found that JAK inhibitors (JAKIs) can effectively relieve and treat a variety of inflammatory skin diseases, including AD, psoriasis, and vitiligo.

JAKIs are small-molecule targeted drugs working intracellularly. When administered, JAKIs can block the actions of a series of cytokines by inhibiting JAKs, thereby inhibiting the activity of JAKs. The first-generation JAKIs include tofacitinib, oclacitinib, baricitinib, and ruxolitinib, which are non-selective and can block various JAKs. Due to the poor selectivity and notable side effects of the first-generation JAKIs, patients taking the first-generation JAKIs undergo many adverse reactions, such as infection, anemia, neutropenia, lymphopenia, cardiovascular diseases, gastrointestinal perforation, and hyperlipidemia. The second-generation JAKIs include Fedratinib, filgotinib, upadacitinib, and abrocitinib. The second-generation JAKIs exhibit high selectivity for JAKs, can merely inhibit a narrow range of cytokines, has enhanced therapeutic targetability, and cause few adverse reactions. When used topically in inflammatory skin diseases, JAKIs can often avoid the systemic adverse reactions.

Proteolysis targeting chimera (PROTAC) is a bifunctional molecule composed of the following three parts: a ligand for binding to a target protein, a ligand for an E3 ubiquitin ligase, and a linker connecting the two. One end of PROTAC can bind to a target protein and the other end of PROTAC can bind to an E3 ligase to produce a ternary complex. The E3 ligase and the target protein are spatially adjacent to each other. Thus, the E3 ligase can mediate the ubiquitination of the target protein with an E2 ubiquitin-conjugating enzyme. Then, a proteasome recognizes and degrades the ubiquitinated target protein. Theoretically, the PROTAC molecules simply provide a binding activity, have an event-driven mode of action, and do not require the long-term high-strength binding to a target protein. Therefore, the PROTAC molecules can be recycled repeatedly. Compared with the occupancy-driven traditional drugs, the PROTAC molecules have the following advantages: (1) The PROTAC molecules provide a novel rapid and reversible chemical knockout method. (2) The PROTAC molecules achieve the druggability of undruggable targets. (3) The PROTAC molecules can reduce the drug toxicity and side effects. (4) The PROTAC molecules can maintain the long-term sensitivity of clinical resistant mutation-prone targets. (5) The PROTAC molecules can affect the non-enzymatic activity function by degrading the whole protein.

The PROTAC molecules combine the advantages of a small-molecule compound and RNA interference. The PROTAC molecules can not only effectively target a target protein, but also degrade and eliminate the target protein, which provides an ideal tool for the effective blockage of key targets in inflammatory diseases such as JAK. After entering the cells, JAK-PROTACs degrade JAKs without relying on the long-term drug concentration pressure, which overcomes the problem that it is difficult to maintain a high drug concentration in a skin tissue for a long time during the topical medication, and especially can avoid the consistent drawback that the topical application of a small-molecule inhibitor to the skin is difficult to achieve an ideal therapeutic effect due to factors such as patient compliance, clothing, and sweat.

The patent application CN115073543A, published on Sep. 20, 2022, discloses a prodrug for JAKI and preparation and use thereof. The JAKI can be used for treating gastrointestinal inflammatory diseases. The commercially-available JAKIs, such as baricitinib and ruxolitinib, can treat an inflammatory skin disease by inhibiting one or more JAKs.

JAK plays an important role in immune-mediated inflammatory diseases, especially inflammatory skin diseases. Therefore, the blockage of JAK is of great significance to the treatment of the above diseases. There have been no reports of using compounds like the JAK-PROTAC-containing compound of the present disclosure for inflammatory skin diseases.

### SUMMARY

A first objective of the present disclosure is to provide a compound in view of the defects of the treatment of inflammatory skin diseases in the prior art.

A second objective of the present disclosure is to provide a pharmaceutical composition.

A third objective of the present disclosure is to provide a JAK-PROTAC compound-containing pharmaceutical composition.

A fourth objective of the present disclosure is to provide a method for preparing the pharmaceutical composition.

A fifth objective of the present disclosure is to provide a use of the pharmaceutical composition.

To achieve the first objective, the present disclosure adopts the following technical solution:

A compound is provided, where the compound includes a JAK-PROTAC compound and a pharmaceutically acceptable salt thereof shown in a Formula (I) (Compound 1 to Compound 4):

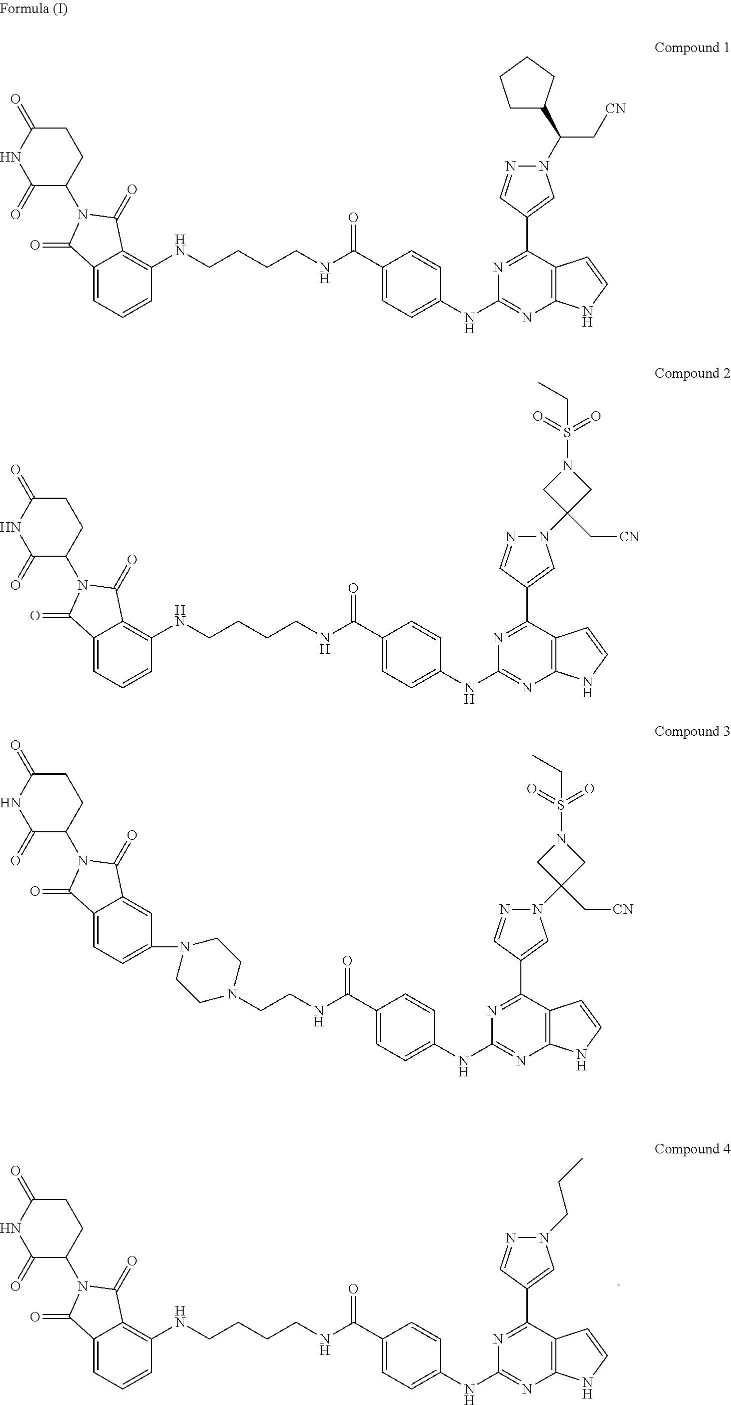 [SMILES: N#CC[C@H](C1CCCC1)n1cc(-c2nc(Nc3ccc(C(=O)NCCCCNc4cccc5c4C(=O)N(C4CCC(=O)NC4=O)C5=O)cc3)nc3[nH]ccc23)cn1 ; CCS(=O)(=O)N1CC(CC#N)(n2cc(-c3nc(Nc4ccc(C(=O)NCCCCNc5cccc6c5C(=O)N(C5CCC(=O)NC5=O)C6=O)cc4)nc4[nH]ccc34)cn2)C1 ; CCS(=O)(=O)N1CC(CC#N)(n2cc(-c3nc(Nc4ccc(C(=O)NCCN5CCN(c6ccc7c(c6)C(=O)N(C6CCC(=O)NC6=O)C7=O)CC5)cc4)nc4[nH]ccc34)cn2)C1 ; CCCn1cc(-c2nc(Nc3ccc(C(=O)NCCCCNc4cccc5c4C(=O)N(C4CCC(=O)NC4=O)C5=O)cc3)nc3[nH]ccc23)cn1]

To achieve the second objective, the present disclosure adopts the following technical solution:

A pharmaceutical composition is provided, including the above-defined compound as an active ingredient or a main active ingredient and a pharmaceutically acceptable carrier, where a dosage form of the pharmaceutical composition includes pharmaceutically acceptable excipient, diluent, or supporting agent.

As a preferred embodiment, a concentration of the compound or the pharmaceutically acceptable salt thereof in a treatment system is 0.01 μM to 1,000 μM.

To achieve the third objective, the present disclosure adopts the following technical solution:

A JAK-PROTAC compound-containing pharmaceutical composition is provided.

Preferably, in weight percentages, the JAK-PROTAC compound-containing pharmaceutical composition includes: an active ingredient: 0.05% to 5%; an oil-phase solid ingredient: 10% to 15%; a permeation enhancer: 0.5% to 5%; a consistency-regulating agent: 15% to 30%; a humectant: 3% to 10%; an emulsifier: 0.5% to 5%; a preservative: 0.05% to 1%; a pH-regulating agent sufficient to adjust a pH value to 6.5 to 8.0; and balanced purified water.

Preferably, an oil phase in the oil-phase solid ingredient is one or more selected from the group consisting of stearic acid, glyceryl monostearate, glyceryl distearate, cetyl alcohol, and stearyl alcohol; the permeation enhancer is laurocapram; the consistency-regulating agent is one or more selected from the group consisting of white petrolatum, liquid paraffin, and 1,3-propanediol; the humectant is one or two selected from the group consisting of glycerol and propylene glycol; the emulsifier is one or two selected from the group consisting of polyoxyethylene-21 stearyl alcohol ether (brij 721) and polyoxyethylene-2 stearyl alcohol ether (brij 72); the pH-regulating agent is one or more selected from the group consisting of triethanolamine, sodium hydroxide, and sodium bicarbonate; and the preservative is one or more selected from the group consisting of chlorobutanol, sodium benzoate, and ethyl p-hydroxybenzoate.

Preferably, the oil phase in the oil-phase solid ingredient is glyceryl monostearate and cetyl alcohol; and the consistency-regulating agent is white petrolatum and liquid paraffin.

To achieve the fourth objective, the present disclosure adopts the following technical solution.

A method for preparing the JAK-PROTAC compound-containing pharmaceutical composition is provided, including the following steps:
a) crushing the active ingredient of the JAK-PROTAC compound-containing pharmaceutical composition to a particle size of 10 μm or less;
b) dissolving and thoroughly mixing a solid in the oil-phase solid ingredient and the consistency-regulating agent at the prescribed amounts to produce an oil phase B;
c) taking the prescribed amounts of the emulsifier, the humectant, the preservative, and the active ingredient, adding the purified water, thoroughly mixing, stirring to allow complete dissolution, and adding the pH-regulating agent to produce an aqueous phase A; and
d) pouring the aqueous phase A into the oil phase B under stirring, and condensing into a paste to produce the JAK-PROTAC compound-containing pharmaceutical composition.

Preferably, a reaction between the solid in the oil-phase solid ingredient and the consistency-regulating agent in the step b) and a reaction of the emulsifier, the humectant, the preservative, the active ingredient, and the purified water in the step c) both are conducted at 70 °C to 85 °C; a pH range adjusted by the pH-regulating agent in the step c) is 6.5 to 8.0; and a reaction between the aqueous phase and the oil phase in the step d) is conducted at 60 °C to 80 °C.

To achieve the fifth objective, the present disclosure adopts the following technical solution.

A use of the compound described above or the pharmaceutical composition described above in preparation of a product for improving or treating an inflammatory skin disease by inhibiting JAK is provided.

Preferably, the product is a drug, a health care product, or a skin care product.

The present disclosure has the following advantages:

1. JAKI based on PROTAC is synthesized in the present disclosure. The JAKI can block the signaling of the JAK pathway through the targeted degradation of JAK, so as to alleviate or treat inflammation and related diseases.

2. Compared with the dexamethasone cream, the JAK-PROTAC compounds have been found to exhibit a very prominent therapeutic effect in AD animal models, especially in the improvement of edema, scales, pruritus, etc. Moreover, the JAK-PROTAC compounds have a JAK degradation activity, and can significantly target the degradation of the target protein JAK. Therefore, the JAK-PROTAC compounds have the potential to be used for treating AD or related skin diseases involved by JAK, which provides a new application direction for the JAK-PROTAC compounds.

### Terms of the Present Disclosure:

To facilitate the understanding of the present disclosure, the present disclosure will be fully described below, and the preferred embodiments of the present disclosure are given below. However, the present disclosure can be embodied in various different forms, and is not limited to the embodiments set forth herein. On the contrary, these embodiments are provided such that the disclosure of the present disclosure can be understood thoroughly and comprehensively. Unless otherwise defined, all technical and scientific terms used herein have the same meanings as commonly understood by those skilled in the technical field of the present disclosure. The terms used in the specification of the present disclosure herein are only for the purpose of describing specific embodiments, and are not intended to limit the present disclosure. The term “and/or” used herein includes any and all combinations of one or more related items listed.

The JAK involves non-receptor tyrosine kinases that transduce cytokine-mediated signals through the JAK-STAT pathway. Janus kinase is short for JAK. It is known that the JAK family mainly includes the four members: JAK1, JAK2, JAK3, and TYK2. The human JAK1 is encoded by a gene JAK1, the human JAK2 is encoded by a gene JAK2, the human JAK3 is encoded by a gene JAK3, and the human TYK2 is encoded by a gene TYK2.

The JAK-PROTAC compound or the pharmaceutically acceptable salt thereof can significantly target the degradation of a target protein JAK, and can degrade JAK through the ubiquitin-proteasome pathway to block the JAK-STAT inflammatory signaling pathway.

The inflammatory skin diseases include skin diseases manifested as local inflammation involved by an immune system or caused by the abnormality of an immune system, including, but not limited to, AD, psoriasis, vitiligo, alopecia areata, skin allergies, etc.

The carrier includes a diluent, an excipient, a filler, a binder, a wetting agent, a disintegrating agent, an absorption enhancer, a surfactant, an adsorption carrier, and a lubricant that are conventional in the pharmaceutical field.

The dosage form is any one selected from the group consisting of a cream, a liniment, a solution, a lyophilized powder for injection, or an injection. Drugs of the above various dosage forms can be prepared according to the conventional method in the pharmaceutical field.

The “pharmaceutically acceptable” refers to those ligands, materials, compositions, and/or dosage forms that are suitable for the administration to a patient within a reasonable medical determination range and match a reasonable benefit/risk ratio.

The “pharmaceutically acceptable carrier” refers to a pharmaceutically acceptable material, composition, or medium, such as a liquid or solid filler, a diluent, an excipient, a solvent, or an encapsulating material. As used herein, the term “pharmaceutically acceptable carrier” includes a buffer, sterile water for injection, a solvent, a dispersion medium, a coating, antibacterial and antifungal agents, an isotonic agent, an absorption-retarding agent and the like that are compatible with the drug administration. In the sense that it is compatible with other ingredients in the formulation and harmless to a patient, each carrier must be pharmaceutically acceptable. Suitable examples include, but are not limited to: (1) sugars, such as lactose, glucose, and sucrose; (2) starches, such as corn starch, potato starch, and substituted or unsubstituted β-cyclodextrin; (3) cellulose and derivatives thereof, such as sodium carboxymethylcellulose, ethylcellulose, and cellulose acetate; (4) powdered tragacanth gum; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) diols, such as propylene glycol; (11) polyols, such as glycerol, sorbitol, mannitol, and polyethylene glycol (PEG); (12) esters, such as ethyl oleate and ethyl laurate; (13) agar; (14) buffers, such as magnesium hydroxide and aluminum hydroxide; (15) alginic acid; (16) pyrogen-free water; (17) isotonic saline; (18) Ringer's solution; (19) ethanol; (20) phosphate buffered saline; and (21) other non-toxic compatible substances adopted in the pharmaceutical formulation.

The “pharmaceutically acceptable salt” refers to a salt that is produced from any compound of the above structure and an acid or alkali and can be used as a drug. The pharmaceutically acceptable salt includes an inorganic salt and an organic salt. One class of salts are salts produced from the compound of the present disclosure and an acid. Acids suitable for the production of salts include, but are not limited to, inorganic acids such as hydrochloric acid, hydrobromic acid, hydrofluoric acid, sulfuric acid, nitric acid, and phosphoric acid; organic acids such as formic acid, acetic acid, trifluoroacetic acid, propionic acid, oxalic acid, malonic acid, succinic acid, fumaric acid, maleic acid, lactic acid, malic acid, tartaric acid, citric acid, picric acid, benzoic acid, methanesulfonic acid, ethanesulfonic acid, p-toluenesulfonic acid, benzenesulfonic acid, and naphthalenesulfonic acid; and amino acids such as proline, phenylalanine, aspartic acid, and glutamic acid. Another class of salts are salts produced from the compound of the present disclosure and an alkali. Alkalies suitable for the production of salts include, but are not limited to: alkali metal salts (such as sodium or potassium salts); alkaline-earth metal salts (such as magnesium or calcium salts); ammonium salts (such as low-grade ammonium alkyl alcohol salts and other pharmaceutically acceptable amine salts), such as a methylamine salt, an ethylamine salt, a propylamine salt, a dimethylamine salt, a trimethylamine salt, a diethylamine salt, a triethylamine salt, a tert-butylamine salt, an ethylenediamine salt, a hydroxyethylamine salt, a dihydroxyethylamine salt, and a trihydroxyethylamine salt; and amine salts produced from morpholine, piperazine, and lysine.

In an embodiment, a pharmaceutically acceptable adjuvant includes a surfactant. Further, the surfactant is at least one selected from the group consisting of sodium dodecyl sulfonate, sodium dodecyl sulfate (SDS), sodium lauryl sulfate (SLS), a polyoxyethylene sorbitan long-chain fatty acid ester, tocopherol polyethylene glycol 1000 succinate (TPGS-1000), a bile salt, sodium deoxycholate, sodium glycocholate, and polyoxyethylene polyoxypropylene glycol. Further, the surfactant is TPGS-1000.

In an embodiment, the drug further includes a polymer carrier. The polymer carrier is at least one selected from the group consisting of a polyvinyl caprolactam-polyvinyl acetate-polyethylene glycol graft copolymer (Soluplus), hydroxypropyl methylcellulose (HPMC), PEG, chitosan, polyvinylpyrrolidone (PVP), PVP/vinyl acetate (VA), hydroxypropyl cellulose (HPC), hydroxypropyl methylcellulose acetate succinate (HPMCAS), eudragit E100, and cationic copolymers based on dimethylaminoethyl methacrylate, butyl methacrylate, and methyl methacrylate. Further, the polymer carrier is a polyvinyl caprolactam-polyvinyl acetate-polyethylene glycol graft copolymer (Soluplus).

### BRIEF DESCRIPTION OF THE DRAWINGS

FIG. 1 shows SCORing Atopic Dermatitis (SCORAD) results for skin damages in ear application regions of mice in each group.

FIG. 2 shows ear thickness variations of mice in each group.

FIG. 3 shows changes in serum thymic stromal lymphopoietin (TSLP) levels detected by enzyme-linked immunosorbent assay for mice in each group.

FIG. 4A-FIG. 4B show impacts of JAK-PROTAC compounds on a viability of HaCaT cells that are evaluated by cell counting kit-8 (CCK-8) assay.

FIG. 5A-FIG. 5B show impacts of JAK-PROTAC compounds on RAW 264.7 cells that are evaluated by nitric oxide (NO) content assay.

FIG. 6 shows images of ear tissues of eczema model mice in Example 2.

FIG. 7 shows images of ear tissue sections stained with Hematoxylin and Eosin (H&E) for animals in each group.

FIG. 8 shows images of ear tissue sections stained with toluidine blue for animals in each group.

FIG. 9 shows images of spleen tissues of eczema model mice in Example 2.

### DETAILED DESCRIPTION OF THE EMBODIMENTS

It should be noted that the compound of the present disclosure is a JAK-PROTAC compound developed by the Charles G Mullighan's research group. The present disclosure will be further described below with reference to specific implementations. It should be understood that these embodiments are only intended to describe the present disclosure, rather than to limit the scope of the present disclosure. In addition, it should be understood that various changes and modifications may be made to the present disclosure by those skilled in the art after reading the content of the present disclosure, and these equivalent forms also fall within the scope defined by the appended claims of the present disclosure.

### Example 1 Preparation of JAK-PROTAC Compounds

The present disclosure is described in detail below in conjunction with Example 1.

The following preparation method is adopted in all of the following examples:

Step 1: A JAK-PROTAC compound, namely, a compound shown in Formula (I) (Compound 1 to compound 4), was crushed to a particle size of 10 μm or less.

Step 2: Prescribed amounts of a solid in an oil-phase solid ingredient and a consistency-regulating agent were dissolved and mixed at 70 °C to 85 °C to prepare an oil phase A.

Step 3: Prescribed amounts of an emulsifier, a humectant, and a preservative were taken, and purified water was added to produce a mixture. The mixture was thoroughly mixed and heated to 70 °C to 85 °C. A prescribed amount of the JAK-PROTAC compound was added, and stirring was conducted to allow complete dissolution. A pH of a main drug solution was adjusted with a pH-regulating agent to 6.5 to 8.0 to produce an aqueous phase B.

Step 4: At 60 °C to 80 °C, the aqueous phase B was poured into the oil phase A under stirring to produce a mixed phase. The mixed phase was condensed into a paste to produce a final product. The raw material proportions of the present disclosure and the prescribed proportions in Example 1 are shown in Table 1 below:

TABLE 1
Active ingredients, excipients and prescriptions for Example 1

| Proportion (W/W) | Active ingredient and raw material | Amount in Example 1 | |
|---|---|---|---|
| 0.05%-2% | JAK-PROTAC compound | 5 | g |
| 10%-15% | Glyceryl monostearate/glyceryl distearate | 90 | g |
| | Cetyl alcohol/stearyl alcohol | 150 | g |
| 15%-30% | White petrolatum | 180 | g |
| | Liquid paraffin | | |
| 3%-10% | Glycerol | 50 | g |
| | 1,3-propanediol | 60 | mL |
| 0.5%-5% | Laurocapram | 60 | g |
| 0.5%-5% | Polyoxyethylene-21 stearyl alcohol ether | 60 | g |
| | (brij 721) | | |
| | Polyoxyethylene-2 stearyl alcohol ether | | |
| | (brij 72) | | |
| 0.05%-1% | Chlorobutanol | 60 | g |

| | | |
|---|---|---|
| | Sodium benzoate | |
| | Ethyl p-hydroxybenzoate | |
| 0.5%-5% | Laurocapram | |
| Appropriate amount | Triethanolamine, sodium hydroxide, and | |
| | sodium bicarbonate | |
| Adding to 1,000 g | Purified water | Adding to 1,000 mL |

Other compositions were prepared with reference to the above preparation method.

### Example 2 Impacts of JAK-PROTAC Compounds on a Mouse Ear Eczema Model

### 1. Experimental Method

### 1.1 Materials

(1) Preparation method of a JAK-PROTAC cream: the same as in Example 1.

(2) Positive therapeutic drug: a 0.050% dexamethasone acetate cream (manufacturer: Shanghai Skin Disease Hospital).

(3) Blank matrix: a matrix component produced after the active ingredient JAK-PROTAC was removed from the cream.

(4) Molding agent: solution of calcipotriol in ethanol (manufacturer: MACKLIN).

(5) Experimental animals: 30 female SPF-grade BALB/c mice, which each had a body weight of 23 g to 25 g, each were 8 weeks old, and available from Shanghai SiPeiFu Biotechnology Co., Ltd.

### 1.2 Animal Grouping and Treatment

(1) The mice were randomly divided into a blank control group (6 mice) (Control), a model group (6 mice) (Model), a positive drug group (6 mice) (Positive), a Compound 1-treating group (0.15% JAK-PROTAC cream 1 for external use) (6 mice) (Treat 1), and a Compound 2-treating group (0.15% JAK-PROTAC cream 2 for external use) (6 mice) (Treat 2).

(2) Molding was conducted in the following manner:

A. On day 0 to day 7 of the modeling: At 9 a.m., the blank control group was administered with the cream matrix, the model group was administered with the molding agent, the positive group was administered with the molding agent, the Compound 1-treating group was administered with the molding agent, and the Compound 2-treating group was administered with the molding agent. At 5 p.m., the blank control group was not treated, the model group was administered with the cream matrix, the positive group was administered with the dexamethasone acetate cream, the Compound 1-treating group was administered with the JAK-PROTAC cream 1, and the Compound 2-treating group was administered with the JAK-PROTAC cream 2.

B. On day 8 to day 10 of the modeling, at 9 a.m., the blank control group was administered with the cream matrix, the model group was not treated, the positive group was not treated, the Compound 1-treating group was not treated, and the Compound 2-treating group was not treated. At 5 p.m., the blank control group was not treated, the model group was administered with the cream matrix, the positive group was administered with the dexamethasone acetate cream, the Compound 1-treating group was administered with the JAK-PROTAC cream 1, and the Compound 2-treating group was administered with the JAK-PROTAC cream 2.

C. On day 11 of the modeling: At 9 a.m., the blank control group was administered with the cream matrix, the model group was not treated, the positive group was not treated, the Compound 1-treating group was not treated, and the Compound 2-treating group was not treated. At 1 p.m., the mice were sacrificed through cervical dislocation, and the spleen and two ears of each mouse were collected.

(3) Spleen index testing:

After the mice were sacrificed, a spleen tissue was collected, dried with a filter paper to remove blood stains on a surface, and weighed for a wet weight. A spleen index was calculated as follows: spleen index=10,000×spleen weight (g)/body weight (g).

### 1.3 Observation of Indicators

(1) The photographing was conducted every day. The severity of skin damage in an ear application region was evaluated through SCORAD for mice. There were three aspects of dorsal skin damages: skin damage area, skin damage severity (including erythema, edema, oozing or crusting, epidermal exfoliation, skin thickening, and dry skin), and pruritus. Each symptom was scored as 0 to 3 depending on the severity of a skin damage: asymptomatic: 0, mild: 1, moderate: 2, and severe: 3. SCORAD score=skin damage area score/5+7×severity score/2+subjective symptom score. SCORAD: mild: 0 to 24, moderate: 25 to 50, severe: higher than 50, and the highest score: 103.

(2) A body weight and thicknesses of two ears of each mouse were recorded daily.

(3) After the experiment was completed, a spleen index of each mouse was calculated.

(4) The TSLP content in serum was detected by enzyme-linked immunosorbent assay.

### 2. Statistics and Analysis

The data of the present disclosure was processed with the Excel and GraphPad software. The experimental data was tested by one-way ANOVA. The post-hoc multiple comparison was conducted with the least significant difference (LSD). P<0.05 indicated a significant difference.

### 3. Experimental Results

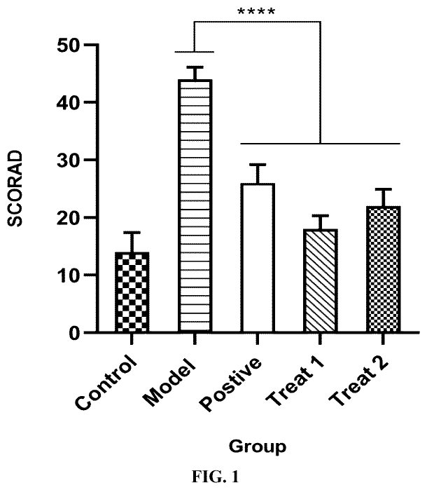

(1) SCORAD was conducted for skin damages in ear application regions of mice daily, as shown in FIG. 1. The erythema, scale, and infiltration scores and the total scores (erythema+scale+infiltration) of mice in the model group all were significantly higher than the respective scores in the Compound 1- and Compound 2-treating groups (P<0.05). These results indicated that the JAK-PROTAC cream could significantly inhibit the inflammatory responses in atopic dermatitis mouse models.

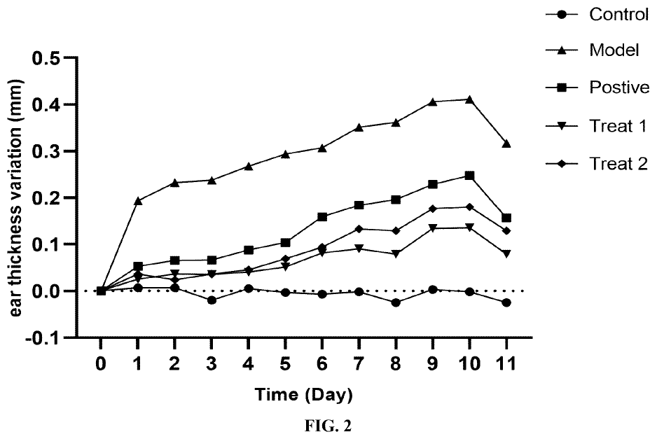

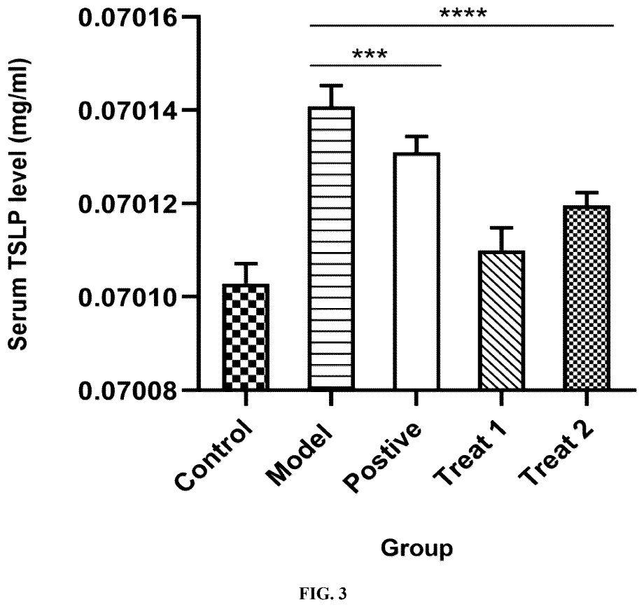

(2) Comparison of ear thicknesses of mice: Before the modeling, there was no significant difference in an ear thickness among groups (P>0.05). After the modeling, ear thicknesses of mice in each group were shown in Table 2, and ear thickness variations were shown in FIG. 2. An ear thickness of the model group was significantly larger than ear thicknesses of the Compound 1- and Compound 2-treating groups, the positive drug group, and the blank control group (P<0.05), and there was no significant difference among the Compound 1- and Compound 2-treating groups and the positive drug group (P>0.05).

(3) Test results of spleen indexes are shown in Table 3. It can be seen from Table 3 that, compared with the blank control group, the spleen weights and spleen indexes of mice in the AD model group, the positive drug group, the Compound 1-treating group, and the Compound 2-treating group all were significantly reduced (P<0.05). In terms of the spleen weight and spleen index, the AD model group, the positive drug group, the Compound 1-treating group, and the Compound 2-treating group were ranked as follows: the Compound 1-treating group>the Compound 2-treating group>the positive drug group>the AD model group (P<0.05). These results indicated that the compounds 1 and 2 had a therapeutic effect for AD, could improve the inflammatory responses in atopic dermatitis mouse models, and exhibited a better therapeutic effect than the positive drug.

(4) Serum TSLP contents of mice in each group were determined by enzyme-linked immunosorbent assay and compared. Results showed that the serum TSLP contents of mice in the model group, the positive drug group, the Compound 1-treating group, and the Compound 2-treating group increased compared with the blank group (P<0.01), the serum TSLP contents of mice in the positive drug group, the Compound 1-treating group, and the Compound 2-treating group were significantly reduced compared with the model group (P<0.01), and reduction levels of TSLP contents in the Compound 1-treating group and the Compound 2-treating group were greater than a reduction level of a TSLP content in the positive group (P<0.01).

TABLE 2
Ear thicknesses of each group of mice (mm)

| Group | Time | Day0 | | Day 1 | | Day2 | | Day3 | | Day4 | |
|---|---|---|---|---|---|---|---|---|---|---|---|
| Blank group | 1 | 0.25 | 0.24 | 0.25 | 0.25 | 0.24 | 0.26 | 0.21 | 0.21 | 0.24 | 0.25 |
| | 2 | 0.22 | 0.22 | 0.21 | 0.22 | 0.22 | 0.22 | 0.20 | 0.19 | 0.23 | 0.24 |
| | 3 | 0.23 | 0.19 | 0.22 | 0.24 | 0.23 | 0.22 | 0.20 | 0.22 | 0.21 | 0.21 |
| | Mean value | 0.23 | | 0.23 | | 0.23 | | 0.21 | | 0.23 | |
| | Standard deviation | 0.02 | | 0.02 | | 0.01 | | 0.01 | | 0.02 | |
| Model group | 1 | 0.25 | 0.27 | 0.27 | 0.33 | 0.32 | 0.31 | 0.30 | 0.33 | 0.32 | 0.31 |
| | 2 | 0.24 | 0.26 | 0.28 | 0.33 | 0.28 | 0.34 | 0.31 | 0.32 | 0.36 | 0.33 |
| | 3 | 0.23 | 0.26 | 0.27 | 0.30 | 0.33 | 0.30 | 0.31 | 0.32 | 0.34 | 0.33 |
| | 4 | 0.22 | 0.25 | 0.27 | 0.35 | 0.29 | 0.33 | 0.31 | 0.31 | 0.35 | 0.34 |
| | Mean value | 0.25 | | 0.30 | | 0.31 | | 0.31 | | 0.34 | |
| | Standard deviation | 0.02 | | 0.03 | | 0.02 | | 0.01 | | 0.02 | |
| Positive | 1 | 0.21 | 0.22 | 0.26 | 0.28 | 0.23 | 0.24 | 0.23 | 0.23 | 0.26 | 0.25 |
| group | 2 | 0.20 | 0.21 | 0.23 | 0.19 | 0.21 | 0.23 | 0.25 | 0.25 | 0.26 | 0.25 |
| | 3 | 0.21 | 0.21 | 0.25 | 0.31 | 0.23 | 0.25 | 0.23 | 0.23 | 0.28 | 0.28 |
| | 4 | 0.20 | 0.22 | 0.24 | 0.27 | 0.23 | 0.24 | 0.23 | 0.23 | 0.28 | 0.26 |
| | Mean value | 0.21 | | 0.25 | | 0.23 | | 0.24 | | 0.27 | |
| | Standard deviation | 0.01 | | 0.03 | | 0.01 | | 0.01 | | 0.01 | |
| Compound 1- | 1 | 0.23 | 0.24 | 0.26 | 0.29 | 0.30 | 0.30 | 0.30 | 0.31 | 0.27 | 0.33 |
| treating | 2 | 0.24 | 0.26 | 0.26 | 0.28 | 0.27 | 0.28 | 0.26 | 0.28 | 0.31 | 0.30 |
| group | 3 | 0.23 | 0.25 | 0.26 | 0.27 | 0.27 | 0.30 | 0.27 | 0.29 | 0.25 | 0.25 |
| | 4 | 0.25 | 0.24 | 0.26 | 0.26 | 0.24 | 0.27 | 0.24 | 0.27 | 0.27 | 0.28 |
| | Mean value | 0.24 | | 0.27 | | 0.28 | | 0.28 | | 0.28 | |
| | Standard deviation | 0.01 | | 0.01 | | 0.02 | | 0.02 | | 0.03 | |
| Compound 2- | 1 | 0.22 | 0.24 | 0.28 | 0.29 | 0.23 | 0.29 | 0.26 | 0.27 | 0.29 | 0.27 |
| treating | 2 | 0.21 | 0.22 | 0.24 | 0.25 | 0.21 | 0.26 | 0.24 | 0.26 | 0.26 | 0.26 |
| group | 3 | 0.21 | 0.24 | 0.26 | 0.24 | 0.23 | 0.25 | 0.26 | 0.26 | 0.28 | 0.28 |
| | 4 | 0.22 | 0.21 | 0.25 | 0.25 | 0.23 | 0.26 | 0.25 | 0.26 | 0.25 | 0.24 |
| | Mean value | 0.22 | | 0.26 | | 0.25 | | 0.26 | | 0.27 | |
| | Standard deviation | 0.01 | | 0.02 | | 0.02 | | 0.01 | | 0.02 | |
| Group | Time | Day5 | | Day6 | | Day7 | | Day8 | | Da [symbol] | |
| Blank group | 1 | 0.20 | 0.22 | 0.23 | 0.22 | 0.24 | 0.23 | 0.22 | 0.22 | 0.24 | [symbol] |
| | 2 | 0.21 | 0.22 | 0.23 | 0.21 | 0.24 | 0.22 | 0.19 | 0.19 | 0.23 | [symbol] |
| | 3 | 0.23 | 0.25 | 0.21 | 0.21 | 0.19 | 0.22 | 0.19 | 0.19 | 0.18 | [symbol] |
| | Mean value | 0.22 | | 0.22 | | 0.22 | | 0.20 | | 0.23 | [symbol] |
| | Standard deviation | 0.02 | | 0.01 | | 0.02 | | 0.01 | | 0.04 | [symbol] |
| Model group | 1 | 0.35 | 0.39 | 0.45 | 0.45 | 0.40 | 0.49 | 0.54 | 0.50 | 0.50 | [symbol] |
| | 2 | 0.35 | 0.32 | 0.37 | 0.40 | 0.46 | 0.45 | 0.54 | 0.50 | 0.50 | [symbol] |
| | 3 | 0.38 | 0.40 | 0.43 | 0.38 | 0.45 | 0.44 | 0.48 | 0.44 | 0.55 | [symbol] |
| | 4 | 0.29 | 0.33 | 0.38 | 0.39 | 0.38 | 0.38 | 0.47 | 0.48 | 0.50 | [symbol] |
| | Mean value | 0.35 | | 0.41 | | 0.43 | | 0.49 | | 0.50 | [symbol] |
| | Standard deviation | 0.04 | | 0.03 | | 0.04 | | 0.03 | | 0.03 | [symbol] |
| Positive | 1 | 0.29 | 0.29 | 0.32 | 0.32 | 0.33 | 0.34 | 0.39 | 0.33 | 0.38 | [symbol] |
| group | 2 | 0.30 | 0.30 | 0.28 | 0.28 | 0.36 | 0.38 | 0.37 | 0.38 | 0.39 | [symbol] |
| | 3 | 0.29 | 0.29 | 0.30 | 0.29 | 0.35 | 0.38 | 0.34 | 0.34 | 0.38 | [symbol] |
| | 4 | 0.28 | 0.29 | 0.32 | 0.29 | 0.34 | 0.36 | 0.37 | 0.38 | 0.46 | [symbol] |
| | Mean value | 0.29 | | 0.30 | | 0.36 | | 0.36 | | 0.40 | [symbol] |
| | Standard deviation | 0.01 | | 0.02 | | 0.02 | | 0.02 | | 0.03 | [symbol] |
| Compound 1- | 1 | 0.32 | 0.32 | 0.34 | 0.35 | 0.33 | 0.32 | 0.30 | 0.33 | 0.40 | [symbol] |
| treating | 2 | 0.29 | 0.28 | 0.36 | 0.35 | 0.37 | 0.35 | 0.32 | 0.32 | 0.40 | [symbol] |
| group | 3 | 0.28 | 0.30 | 0.31 | 0.30 | 0.36 | 0.33 | 0.35 | 0.37 | 0.43 | [symbol] |
| | 4 | 0.24 | 0.32 | 0.25 | 0.33 | 0.26 | 0.34 | 0.24 | 0.34 | 0.30 | [symbol] |
| | Mean value | 0.29 | | 0.32 | | 0.33 | | 0.32 | | 0.38 | [symbol] |
| | Standard deviation | 0.03 | | 0.03 | | 0.03 | | 0.04 | | 0.04 | [symbol] |
| Compound 2- | 1 | 0.29 | 0.30 | 0.32 | 0.33 | 0.34 | 0.36 | 0.34 | 0.34 | 0.40 | [symbol] |
| treating | 2 | 0.30 | 0.29 | 0.31 | 0.31 | 0.36 | 0.36 | 0.36 | 0.39 | 0.42 | [symbol] |
| group | 3 | 0.28 | 0.31 | 0.32 | 0.33 | 0.36 | 0.34 | 0.34 | 0.36 | 0.38 | [symbol] |
| | 4 | 0.26 | 0.29 | 0.30 | 0.30 | 0.35 | 0.36 | 0.33 | 0.34 | 0.42 | [symbol] |
| | Mean value | 0.29 | | 0.32 | | 0.35 | | 0.35 | | 0.40 | [symbol] |
| | Standard deviation | 0.01 | | 0.01 | | 0.01 | | 0.02 | | 0.02 | [symbol] |

[symbol]

indicates data missing or illegible when filed

TABLE 3
Results of spleen indexes

| | | Body | Spleen | Spleen index/ |
|---|---|---|---|---|
| Group | | weight/g | weight/g | (mg · g⁻¹) |
| Blank group | 1 | 26.8 | 0.1386 | 51.7164 |
| | 2 | 22.3 | 0.1404 | 62.9596 |
| | 3 | 24.8 | 0.1398 | 56.3710 |
| | Mean value | 24.6333 | 0.1396 | 56.6712 |
| | Standard | 1.8409 | 0.0007 | 4.6126 |
| | deviation | | | |
| Model group | 1 | 20.1 | 0.0623 | 30.9950 |
| | 2 | 21.8 | 0.0511 | 23.4404 |
| | 3 | 21.8 | 0.0627 | 28.7615 |
| | Mean value | 21.2333 | 0.0587 | 27.6452 |
| | Standard | 0.8014 | 0.0054 | 3.1689 |
| | deviation | | | |
| Positive group | 1 | 18.7 | 0.0560 | 29.9465 |
| | 2 | 19.3 | 0.0638 | 33.0570 |
| | 3 | 19.8 | 0.0602 | 30.4040 |
| | Mean value | 19.2667 | 0.0600 | 31.1419 |
| | Standard | 0.4497 | 0.0032 | 1.3712 |
| | deviation | | | |
| Compound 1- | 1 | 21.4 | 0.0798 | 37.2897 |
| treating group | 2 | 19.9 | 0.0934 | 46.9347 |
| | 3 | 21.0 | 0.0855 | 40.7143 |
| | Mean value | 20.7667 | 0.0862 | 41.5249 |
| | Standard | 0.6342 | 0.0056 | 3.9923 |
| | deviation | | | |
| Compound 2- | 1 | 20.0 | 0.0749 | 37.4500 |
| treating group | 2 | 19.2 | 0.0739 | 38.4896 |
| | 3 | 20.0 | 0.0708 | 35.4000 |
| | Mean value | 19.7333 | 0.0732 | 37.0946 |
| | Standard | 0.3771 | 0.0017 | 1.2836 |
| | deviation | | | |

In summary, the JAK-PROTAC compounds can alleviate the eczema by inhibiting an inflammatory factor, and can also repair the skin barrier by reducing a transepidermal water loss.

### Example 3 Impacts of JAK-PROTAC Compounds on a Viability of HaCaT Cells Evaluated by CCK-8 Assay

### 1. Experimental Method

### 1.1 Materials

(1) Reagents: compound 1: a compound shown by Compound 1 in Formula (I); compound 2: a compound shown by Compound 2 in Formula (I); Dulbecco's Modified Eagle Medium (DMEM) high-glucose medium: Wuhan Pricella Biotechnology Co., Ltd.; South American fetal bovine serum (FBS) and trypsin: Gibco, USA; penicillin-streptomycin: Hyclone, USA; and CCK-8 kit: Beyotime.

(2) Instruments: CO₂ incubator: HERA cell 240; and microplate reader: Thermo Fisher Scientific, USA.

### 1.2 Operation Steps

The cultured HaCaT cells were counted under a microscope to be about 2×10⁶ cells/mL, then inoculated in a 96-well plate at about 6×10³ cells/well, and incubated overnight in a 37 °C and 5% CO₂ incubator. After cells were adherent, a medium was removed, and 200 μL of each of complete media including compounds 1 and 2 at different concentrations (0.05 μg/mL, 0.1 μg/mL, 0.2 μg/mL, 0.5 μg/mL, 1 μg/mL, 2 μg/mL, 5 μg/mL, 10 μg/mL, and 20 μg/mL) was added to a corresponding well. The 0 μg/mL group was set as a blank control group. Three replicate wells were set for each treatment of the compounds 1 and 2. The plate was incubated in a CO₂ incubator for 24 h. 20 μL of CCK-8 reagent was added to each well, and the plate was incubated in a CO₂ incubator for 1 h and then placed in the microplate reader to read the absorbance at 450 nm. The test was repeated 3 times, and a mean value was taken. The measured absorbance was substituted into a cell viability calculation formula to calculate the cell viability.

### 2. Statistics and Analysis

The data of the present disclosure was processed with the Excel and Graphpad software. The experimental data was tested by one-way ANOVA. The post-hoc multiple comparison was conducted with the LSD. P<0.05 indicated a significant difference.

### 3. Experimental Results

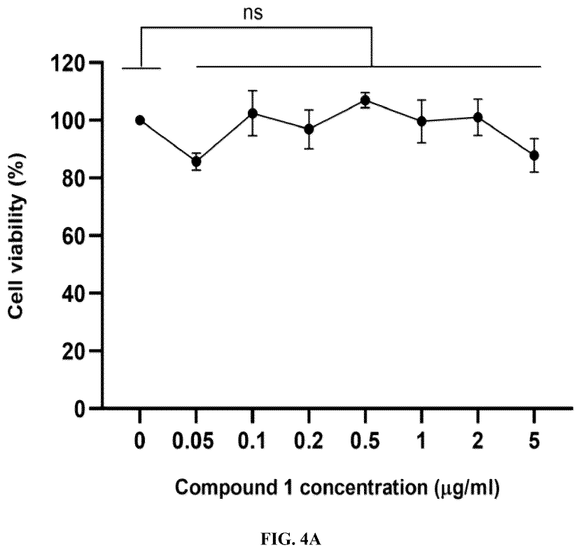

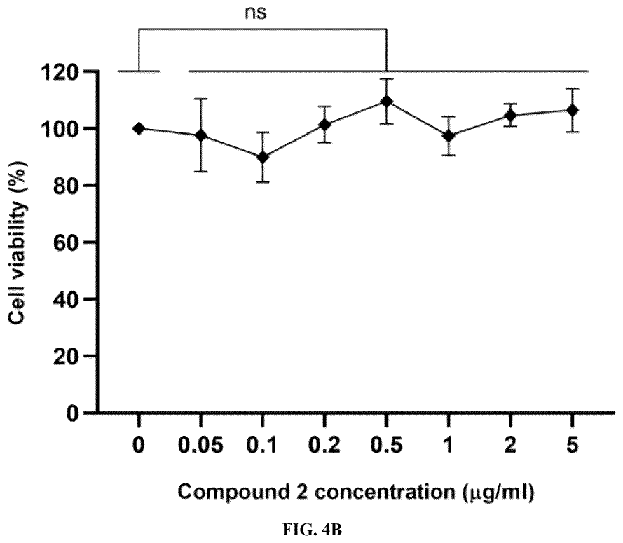

CCK-8 assay results are shown in Table 4 and FIG. 4A-FIG. 4B. The results showed that the cell viability was greater than 80% at compound concentrations of 0.05 μg/mL, 0.1 μg/mL, 0.2 g/mL, 0.5 μg/mL, 1 μg/mL, 2 μg/mL, 5 μg/mL, 10 μg/mL, and 20 μg/mL.

TABLE 4
HaCaT cell viability results determined by CCK-8 assay

| Concentration (μg/mL) | | | | | | | | | |
|---|---|---|---|---|---|---|---|---|---|
| 0.00 | 0.05 | 0.10 | 0.20 | 0.50 | 1.00 | 2.00 | 5.00 | 10.00 | 20.00 |

Compound 1

| | | | | | | | | | | |
|---|---|---|---|---|---|---|---|---|---|---|
| Cell viability (%) | 100.00 | 80.53 | 85.66 | 90.69 | 98.34 | 98.32 | 93.76 | 107.74 | 110.16 | 80.26 |

Compound 2

| | | | | | | | | | | |
|---|---|---|---|---|---|---|---|---|---|---|
| Cell viability (%) | 100.00 | 95.49 | 95.60 | 106.17 | 113.27 | 101.11 | 107.41 | 123.58 | 119.82 | 93.34 |

### Example 4 Impacts of JAK-PROTAC Compounds on RAW 264.7 Cells Evaluated by NO Content Assay

### 1. Experimental Method

### 1.1 Materials

(1) Reagents: compound 1: a compound shown by Compound 1 in formula (I); compound 2: a compound shown by Compound 2 in Formula (I); DMEM high-glucose medium: Wuhan Pricella Biotechnology Co., Ltd.; South American FBS and trypsin: Gibco, USA; penicillin-streptomycin: Hyclone, USA; and nitric oxide (NO) assay kit: Beyotime.

(2) Instruments: CO₂ incubator: HERA cell 240; and microplate reader: Thermo Fisher Scientific, USA.

### 1.2 Operation Steps

Induction of an RAW 264.7 macrophage inflammatory model with lipopolysaccharide (LPS) and interferon-gamma (IFN-γ): RAW 264.7 cells in a logarithmic growth phase were taken and inoculated in a 96-well cell culture plate at 20×10³ cells/well, and cultured for 24 h until the cells were adherent. Ablank control group of 0 μg/mL, a model group of 0.5 μg/mL LPS+2 ng/mL IFN-γ, and experimental groups of compounds 1 and 2 at concentrations of 0.05 μg/mL, 0.1 μg/mL, 0.2 μg/mL, 0.4 μg/mL, and 0.8 μg/mL were set. Three replicate wells were set for each group. In the blank control group, cells were incubated with serum-free DMEM. In the LPS+IFN-γ model group, cells were incubated with serum-free DMEM. After 24 h of incubation, morphological changes of cells were observed, and a concentration of NO was determined according to instructions of the NO assay kit.

### 2. Statistics and Analysis

The data of the present disclosure was processed with the Excel and Graphpad software. The experimental data were tested by one-way ANOVA. The post-hoc multiple comparison was conducted with the LSD. P<0.05 indicated a significant difference.

### 3. Experimental Results

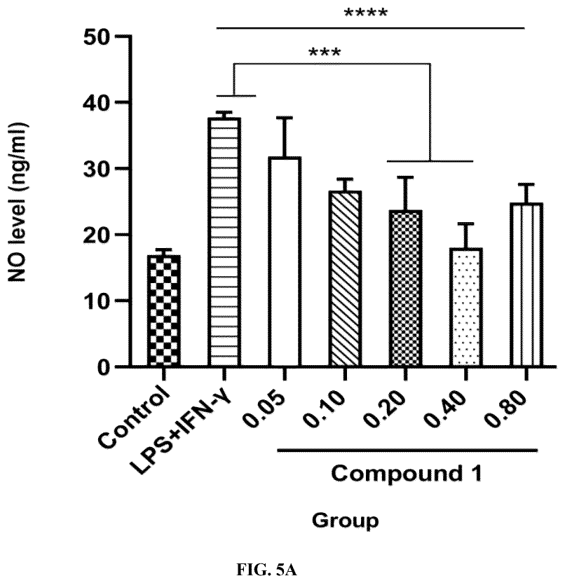

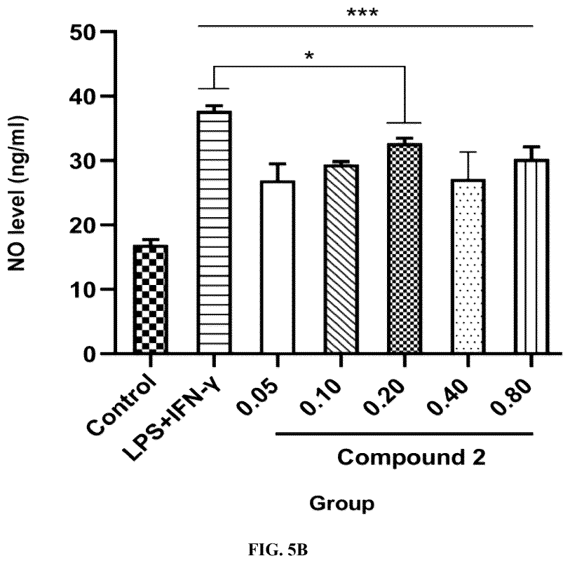

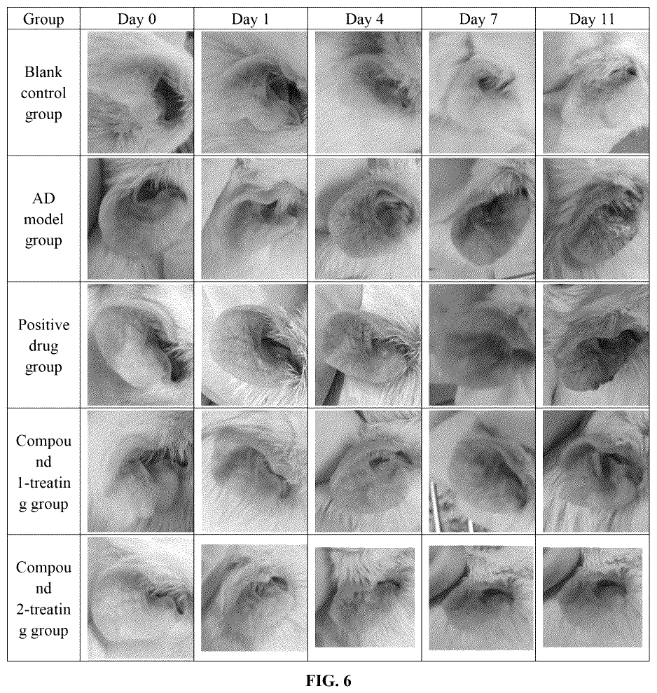

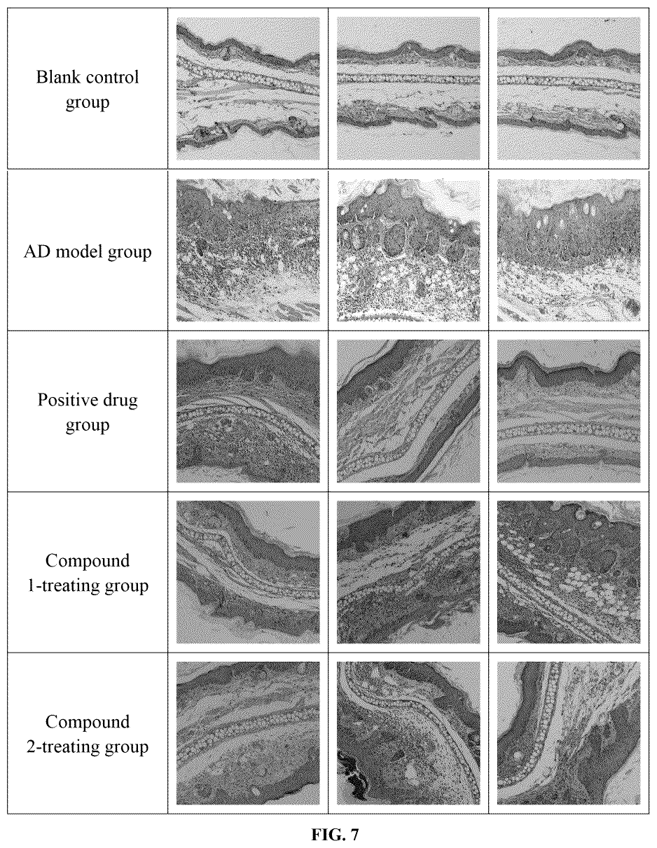

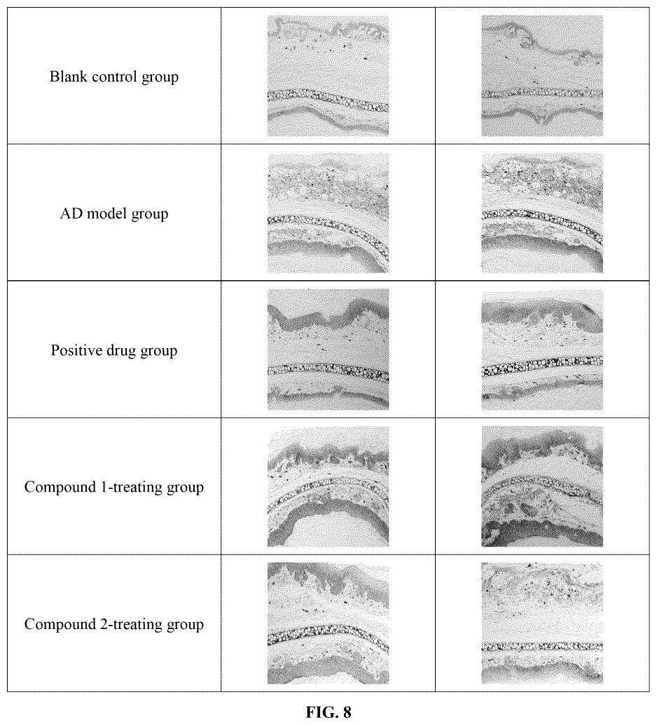

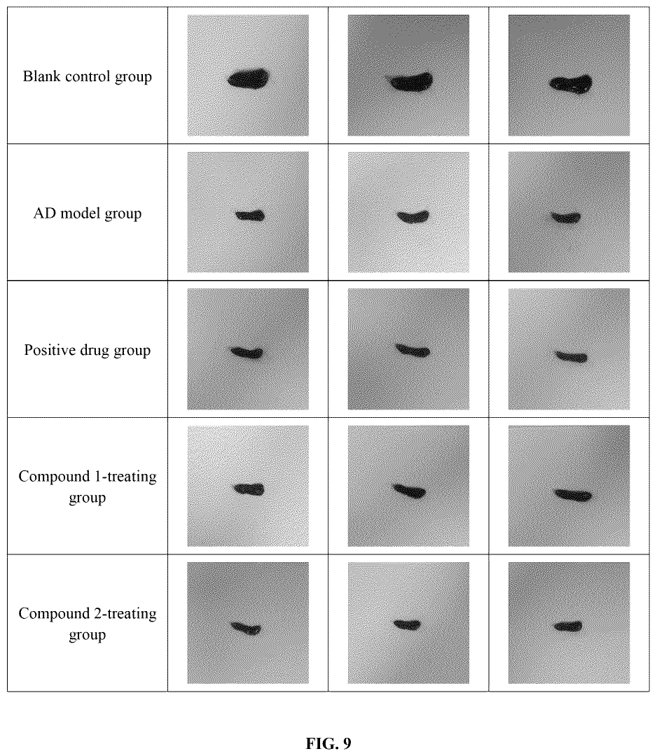

Results of the NO content determination are shown in FIG. 5A and FIG. 5B. As a cytokine in the early stage of acute inflammation, the inflammatory factor NO was adopted to determine whether the inflammatory model was successfully constructed. The inflammatory factor NO could mediate an inflammatory response.

The results showed that an NO level in the LPS+IFN-γ model group was significantly higher than an NO level in the blank control group, indicating that the RAW 264.7 macrophage inflammatory model was successfully induced. Compared with the LPS+IFN-γ model group, NO levels of the Compound 1 and Compound 2 experimental groups of 0.05 μg/mL, 0.1 μg/mL, 0.2 g/mL, 0.4 μg/mL, and 0.8 μg/mL were reduced, indicating that the compounds 1 and 2 in the concentration range of 0.05 μg/mL to 0.8 μg/mL could effectively inhibit the inflammation and exhibited the optimal anti-inflammatory effect at a concentration of 0.4 μg/mL.

### Example 5 Investigation of Therapeutic Effects of JAK-PROTAC Creams for Eczema Subjects

### 1. Method

Two patients with moderate to severe AD were selected as subjects, and treated with a 0.15% JAK-PROTAC cream 1 for external use and a 0.15% JAK-PROTAC cream 2 for external use, respectively.

### 2. Statistics and Analysis

Changes at skin damage sites before a treatment and on day 1, day 4, and day 7 of the treatment were recorded through photographing. Therapeutic effects of the two creams for external use were determined according to damage degrees at skin damage sites.

### 3. Results

The results showed that, compared with the conditions before the treatment, the administration of the 0.15% JAK-PROTAC cream 1 for external use significantly improved the skin damage area, erythema, edema, crusting, epidermal exfoliation, skin thickening, and itching symptoms in the eczema subjects, and the administration of the 0.15% JAK-PROTAC cream 2 for external use significantly improved the skin damage area, skin thickening, and itching symptoms in the eczema subjects. It indicated that the two 0.15% JAK-PROTAC creams for external use both could achieve superior eczema-treating effects.

The above are merely preferred implementations of the present disclosure. It should be noted that a person of ordinary skill in the art may further make several improvements and modifications without departing from the method of the present disclosure, but such improvements and modifications should be deemed as falling within the protection scope of the present disclosure.

1. A compound shown in Formula (I) (Compound 1 to compound 4), wherein the compound comprises a Janus kinase (JAK)-proteolysis targeting chimera (PROTAC) compound and a pharmaceutically acceptable salt thereof:

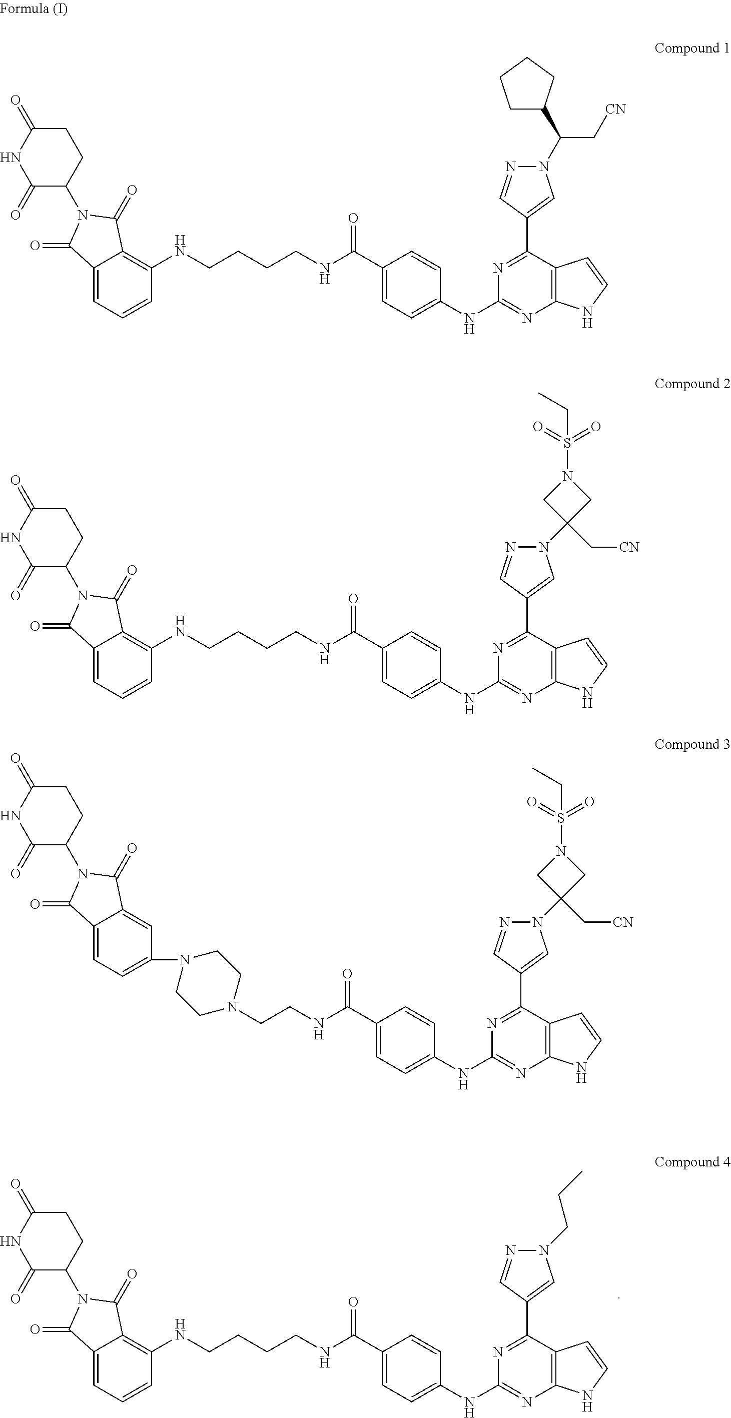 [SMILES: N#CC[C@H](C1CCCC1)n1cc(-c2nc(Nc3ccc(C(=O)NCCCCNc4cccc5c4C(=O)N(C4CCC(=O)NC4=O)C5=O)cc3)nc3[nH]ccc23)cn1 ; CCS(=O)(=O)N1CC(CC#N)(n2cc(-c3nc(Nc4ccc(C(=O)NCCCCNc5cccc6c5C(=O)N(C5CCC(=O)NC5=O)C6=O)cc4)nc4[nH]ccc34)cn2)C1 ; CCS(=O)(=O)N1CC(CC#N)(n2cc(-c3nc(Nc4ccc(C(=O)NCCN5CCN(c6ccc7c(c6)C(=O)N(C6CCC(=O)NC6=O)C7=O)CC5)cc4)nc4[nH]ccc34)cn2)C1 ; CCCn1cc(-c2nc(Nc3ccc(C(=O)NCCCCNc4cccc5c4C(=O)N(C4CCC(=O)NC4=O)C5=O)cc3)nc3[nH]ccc23)cn1]

2. A pharmaceutical composition, comprising the compound according to claim 1 as an active ingredient or a main active ingredient and a pharmaceutically acceptable carrier, wherein a dosage form of the pharmaceutical composition comprises pharmaceutically acceptable excipient, diluent, or supporting agent.

3. The pharmaceutical composition according to claim 2, wherein a concentration of the compound or the pharmaceutically acceptable salt thereof in a treatment system is 0.01 μM to 1,000 μM.

4. A JAK-PROTAC compound-containing pharmaceutical composition prepared with the compound according to claim 1, wherein in weight percentages, the pharmaceutical composition comprises: an active ingredient: 0.05% to 5%; an oil-phase solid ingredient: 10% to 15%; a permeation enhancer: 0.5% to 5%; a consistency-regulating agent: 15% to 30%; a humectant: 3% to 10%; an emulsifier: 0.5% to 5%; a preservative: 0.05% to 1%; a pH-regulating agent sufficient to adjust a PH value to 6.5 to 8.0; and balanced purified water.

5. The pharmaceutical composition according to claim 4, wherein an oil phase in the oil-phase solid ingredient is one or more selected from the group consisting of stearic acid, glyceryl monostearate, glyceryl distearate, cetyl alcohol, and stearyl alcohol; the permeation enhancer is laurocapram; the consistency-regulating agent is one or more selected from the group consisting of white petrolatum, liquid paraffin, and 1,3-propanediol; the humectant is one or two selected from the group consisting of glycerol and propylene glycol; the emulsifier is one or two selected from the group consisting of polyoxyethylene-21 stearyl alcohol ether (brij 721) and polyoxyethylene-2 stearyl alcohol ether (brij 72); the pH-regulating agent is one or more selected from the group consisting of triethanolamine, sodium hydroxide, and sodium bicarbonate; and the preservative is one or more selected from the group consisting of chlorobutanol, sodium benzoate, and ethyl p-hydroxybenzoate.

6. The pharmaceutical composition according to claim 5, wherein the oil phase in the oil-phase solid ingredient is glyceryl monostearate and cetyl alcohol; and the consistency-regulating agent is white petrolatum and liquid paraffin.

7. A method for preparing the pharmaceutical composition according to claim 4, comprising the following steps:
a) crushing the active ingredient of the pharmaceutical composition to a particle size of 10 μm or less;
b) dissolving and thoroughly mixing a solid in the oil-phase solid ingredient and the consistency-regulating agent at the prescribed amounts to produce an oil phase B;
c) taking the prescribed amounts of the emulsifier, the humectant, the preservative, and the active ingredient, adding the purified water, thoroughly mixing, stirring to allow complete dissolution, and adding the pH-regulating agent to produce an aqueous phase A; and
d) pouring the aqueous phase A into the oil phase B under stirring, and condensing into a paste to produce the pharmaceutical composition.

8. The method according to claim 7, wherein a reaction between the solid in the oil-phase solid ingredient and the consistency-regulating agent in the step b) and a reaction of the emulsifier, the humectant, the preservative, the active ingredient, and the purified water in the step c) both are conducted at 70 °C to 85 °C; a pH range adjusted by the pH-regulating agent in the step c) is 6.5 to 8.0; and a reaction between the aqueous phase and the oil phase in the step d) is conducted at 60 °C to 80 °C.

9. A method for preparing a product for improving or treating an inflammatory skin disease by inhibiting JAK, comprising including the compound according to claim 1 into the product.

10. The method according to claim 9, wherein the product is a drug, a health care product, or a skin care product.
